(19) United States
(12) Patent Application Publication
(10) Pub. No.: US 2022/0290883 A1
(43) Pub. Date: Sep. 15, 2022

(54) GERMICIDAL AIR DIFFUSER

(71) Applicant: Rickard Air Diffusion (Pty) Ltd, Ottery (ZA)

(72) Inventor: Mark Gareth RICKARD, Ottery (ZA)

(73) Assignee: Rickard Air Diffusion (Pty) Ltd, Ottery (ZA)

(21) Appl. No.: 17/682,185

(22) Filed: Feb. 28, 2022

(30) Foreign Application Priority Data
Mar. 15, 2021 (ZA) 2021/01710

(51) Int. Cl. F24F 8/22; F24F 13/062; A61L 9/20

(52) U.S. Cl. CPC F24F 8/22; F24F 13/062; A61L 9/20; A61L 2209/12; A61L 2209/16

(57) ABSTRACT

An air diffuser (10) includes a casing (16) with an inlet (18) connected to a supply of air, and an outlet (22) defining a diffusion flow passage extending in a diffusion direction (30). A cover (32) is disposed adjacent the diffusion flow passage to define an induction flow passage (36) that is in flow communication with the diffusion flow passage and is oriented such that air from the induction flow passage (36) is drawn into the diffusion flow passage to be discharged into a room (12). A germicidal light source (38) is provided to irradiate the induction flow passage (36) and kill airborne pathogens.

### RELATED APPLICATION

This application claims the benefit of priority of South Africa Patent Application No. 2021/01710 filed on Mar. 15, 2021, the contents of which are incorporated herein by reference in their entirety.

### FIELD AND BACKGROUND OF THE INVENTION

This invention relates to air diffusers for use in air conditioning systems.

Air diffusers are used to convert a combination of static and velocity pressure to air movement as air is distributed from a heating, ventilation and air conditioning (HVAC) system to a space such as an air-conditioned room. (The space targeted by an HVAC system is referred to as a “room” herein, for the sake of brevity, but the space can take many other forms.)

A need often arises to inactivate or kill airborne microorganisms or pathogens in a room and some of the methods used presently to achieve this, include the use of ultraviolet germicidal irradiation (UVGI) in the form of short wavelength ultraviolet light (UV-C).

One of the present methods of inactivating or killing airborne microorganisms or pathogens in a room uses static UVGI devices that are installed to direct UV-C horizontally to irradiate the upper air in a room, to create an irradiation or disinfection zone above an occupied zone in which occupants are usually safe from the irradiation from the UVGI—although these installations can hold risks of UV-C exposure in the occupied zone, because of the proximity of the irradiation zone. In order for contaminated air to move from the occupied zone to the disinfection zone, such an upper air disinfection systems requires either forced air movement, which requires motive energy and can be noisy or disruptive, or the system can rely on natural air movement such as convection, which is often not effective enough to provide the desired germicidal effect.

Another method of inactivating or killing airborne microorganisms or pathogens in a room, is the use of self-contained disinfection modules that include sources of germicidal UV-C in enclosures, with forced air flow inducers such as fans to circulate air through disinfection zones in the enclosures. Units of this type can be effective in treating contaminated air in a single pass, but their effectiveness in a room depends on their ability to recirculate air in the room—which is achieved by energy-consuming forced air flow induction—such as fans.

The present invention seeks to inactivate or kill airborne microorganisms or pathogens in a room efficiently, while ameliorating the disadvantages of the prior art, mentioned above.

### SUMMARY OF THE INVENTION

According to the present invention there is provided an air diffuser comprising:
a casing defining an inlet that is configured to be connected to a supply of air and an outlet defining a diffusion flow passage extending in a diffusion direction;
a cover disposed adjacent the diffusion flow passage, said cover defining an induction flow passage that is in flow communication with the diffusion flow passage, said induction flow passage being oriented relative to the diffusion direction, such that air from the induction flow passage is drawn into the diffusion flow passage, when air flows in the diffusion flow passage in the diffusion direction; and
a germicidal light source disposed to irradiate at least part of the induction flow passage.

The induction flow passage may be oriented at an acute angle relative to the diffusion direction. Persons skilled in the art would realise that if there is an acute angle between the induction flow passage and the diffusion direction, then there is also an obtuse angle between the induction flow passage and the diffusion direction—depending on which sides of the flow passages the angle is measured.

The air diffuser may define a control flow passage between the inlet and the outlet and the air diffuser may include an air flow control mechanism that is configured to restrict the control flow passage selectively to control the rate at which air can flow from the air supply to the diffusion flow passage.

The cover may include a spaced plate that is spaced from the casing such that the induction flow passage may be defined in the space between the spaced plate and the casing. The spaced plate may be oriented at an acute angle relative to the diffusion direction so that air flowing in the diffusion flow passage impinges on the spaced plate at an acute angle.

The air diffuser may include a central plate and the outlet may extend around the periphery of the central plate, with the diffusion flow passage extending conically outwardly from the central plate. The spaced plate may define a central aperture and may be disposed so that the central aperture is generally aligned with a centre of the central plate and so that the spaced plate is generally in register with the outlet.

### BRIEF DESCRIPTION OF THE SEVERAL VIEWS OF THE DRAWINGS

For a better understanding of the present invention, and to show how it may be carried effect, the invention will now be described by way of non-limiting example, with reference to the accompanying drawings in which:

FIG. 1 shows a three-dimensional view of an air diffuser according to the present invention, viewed from the room below the diffuser; and

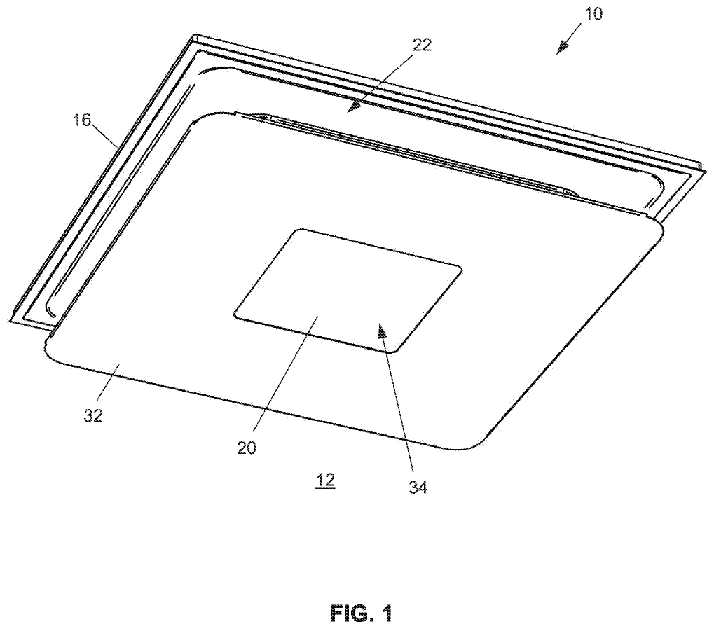

FIG. 2 shows a diagrammatic sectional side view of the diffuser of FIG. 1.

### DESCRIPTION OF SPECIFIC EMBODIMENTS OF THE INVENTION

Referring to the drawings, an air diffuser according to the present invention, is generally identified by reference sign 10 and a space that is targeted by the diffuser is a room that generally identified by reference sign 12.

The diffuser 10 is configured to distribute air from an air supply to the room 12. The air supply could be from various sources, but typically, the air is supplied from an HVAC system and in the illustrated example, the air is supplied to the diffuser 10 via an air duct 14.

The present invention could be used with various configurations of diffusers 10, but in the illustrated example, the diffuser 10 is a variable air volume diffuser that can regulate the flow-rate of air distributed to the room 12 and the flow-rate can be adjusted to achieve desired ventilation and/or temperature conditioning.

The diffuser 10 includes a casing 16 with an inlet 18, where it receives a supply of air from the duct 14. At the bottom of the casing 16, a fixed central plate or trim plate 20 is supported generally flush with the underside of the casing and typically flush with an adjacent ceiling, with an outlet 22 defined around the periphery of the trim plate.

Between the inlet 18 and the outlet 22, the diffuser 10 defines a control flow passage 24 inside the casing 16 and an air flow control mechanism 26 is provided that is configured to restrict the control flow passage selectively to control the rate at which air can flow from the inlet to the outlet. The air flow control mechanism 26 in the illustrated example is automatic and lifts and lowers a control disc 28 to adjust restriction of the control flow passage by adjusting a space between the periphery of the control disc and the casing 16.

At the outlet 22, the diffuser 10 defines a diffusion flow passage, which extends from the circumferences of the control disc 28 and trim plate 20, outwards in a diffusion direction 30. The diffusion flow passage extends outwards from the entire circumference of the trim plate 20 and extends downwards at a small angle (of about 30 degrees in the illustrated example), so that the diffusion flow passage is generally conical.

Below the casing 16 the diffuser 10 includes a cover, which in the illustrated example is a cover plate 32 that defines a central aperture 34 and that is spaced below the casing 16 and trim plate 20. The cover plate 32 is disposed so that the central aperture 34 is generally aligned with a centre of the trim plate 20 and so that the periphery of the cover plate is generally in register with the outlet 22. An induction flow passage 36 is defined between the cover plate 32 and the underside of the trim plate 20. The induction flow passage 36 extends horizontally outwardly from the centre of the trim plate 20 and the aperture 34, between the generally parallel trim plate and cover plate 32 and the induction flow passage meets up with the diffusion flow passage, which extends in the diffusion direction 30.

The diffusion direction 30 extends outwards and downwards, whereas the induction flow passage 36 extend outwards and horizontal, so that the diffusion passage is oriented at an acute angle relative to the diffusion direction and air flowing in the diffusion passage would impinge the top of the cover plate 32 at an obtuse angle, near its periphery.

In other embodiments of the invention it would be possible to configure an induction flow passage that intersects the diffusion flow passage perpendicularly or to configure an induction flow passage that extends parallel to the diffusion flow passage, before the two passages intersect—as long as air flowing in the diffusion direction in the diffusion flow passage induces air to flow in the induction flow passage, towards the diffusion flow passage. However, in preferred embodiments, such as the illustrated embodiment, to the induction flow passage is oriented at an acute angle relative to the diffusion direction, so that air from the induction flow passage is drawn into air flowing outwards in the diffusion flow passage.

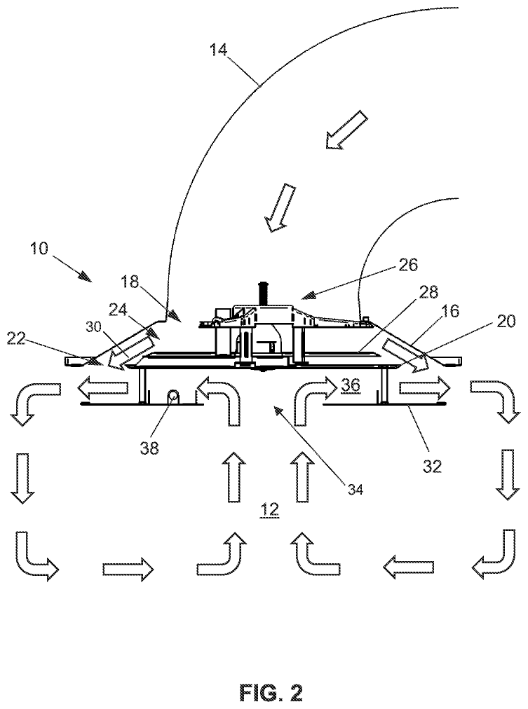

A germicidal light source 38, such as a fluorescent tube emitting UV-C, is provided in the induction flow passage 36, or adjacent the induction flow passage, so that germicidal light from the light source irradiates at least part of the induction flow passage. In FIG. 2, only a single germicidal light source 38 is shown, but the diffusion 10 could include more light sources and in a preferred embodiment, the diffuser includes two diametrically opposed light sources.

In use, when the diffuser 10 is operated to discharge air into the room 12, the air flows along the diffusion flow passage in the diffusion direction 30—as it would in diffusers of the prior art. The angled intersection of the induction flow passage 36 relative to the diffusion direction 30 causes air in the induction flow passage to be drawn into the flow stream of air from the outlet 22, so that the air from the induction flow passage and air from the outlet are combined and flow outward, into the room. A flow of air in the induction passage 36 is thus induced by the angled intersection between the induction flow passage and the diffusion direction.

In other embodiments of the invention, air may be diffused in different diffusers, e.g. air may be discharged in a single direction from a linear diffuser, to provide an air curtain. Air may be diffused from a static diffuser on a side wall of a duct, etc. However, in each of these instances (and countless others), a cover such as a plate or other formation can be provided according to the present invention, to define an induction flow passage that intersects the flow of air from the diffuser outlet, preferably at an acute angle, so that a flow of air is induced in the induction flow passage. The intersection of the induction flow passage and the diffuser outlet is preferably at an acute angle, but it could also be at a right angle.

As air passes through the induction flow passage 36, it is exposed to UV-C irradiation (or other germicidal light, in other embodiments of the invention) from the light source 38 and the irradiation inactivates or kills airborne microorganisms or pathogens in the air flowing in the induction flow passage in an irradiation zone. The air that has been exposed to irradiation from the light source 38 then combines with air from the outlet 22 (as described above) and flow outwards into the room 12. Air is circulated in the room and some air is recycled into the induction flow passage—which in the illustrated example means that air from the room 12 enters the aperture 34 from below and flows outwards in the induction flow passage, where it is exposed to irradiation from the light source 38. The circulation of air in the room is illustrated diagrammatically with arrows in FIG. 2.

The present invention thus continuously draws air from the room 12 into the induction flow passage 36 where it is exposed to germicidal irradiation from the light source 38, before being returned to the room, in combination with air from the diffuser 10. The invention thus achieves effective exposure to high volumes of air from the room 12 and provides effective germicidal irradiation, but it does so cost-effectively, because only relatively simple, static additions to air diffusers of the prior art is required. The present invention also operates efficiently and quietly because it does not require forced air flow induction such as a fan in the room 12, but relies on air flow from the HVAC system to induce air flow through the irradiation zone. Lastly, the irradiation zone in the induction flow passage 36 is shielded very effectively from the room 12 by the cover plate 32, so that the risk of harmful exposure to UV-C irradiation to occupants of the room, is negligible.

What is claimed is:

1. An air diffuser comprising:
a casing defining an inlet that is configured to be connected to a supply of air, and an outlet defining a diffusion flow passage extending in a diffusion direction;
a cover disposed adjacent the diffusion flow passage, said cover defining an induction flow passage that is in flow communication with the diffusion flow passage, said induction flow passage being oriented relative to the diffusion direction, such that air from the induction flow passage is drawn into the diffusion flow passage, when air flows in the diffusion flow passage in the diffusion direction; and
a germicidal light source disposed to irradiate at least part of the induction flow passage.

2. The air diffuser according to claim 1, wherein the induction flow passage is oriented at an acute angle relative to the diffusion direction.

3. The air diffuser according to claim 1, which defines a control flow passage between the inlet and the outlet.

4. The air diffuser according to claim 3, which includes an air flow control mechanism that is configured to restrict the control flow passage selectively to control the rate at which air can flow from the air supply to the diffusion flow passage.

5. The air diffuser according to claim 1, wherein the cover includes a spaced plate that is spaced from the casing such that the induction flow passage is defined in a space between the spaced plate and the casing.

6. The air diffuser according to claim 5, wherein the spaced plate is oriented at an acute angle relative to the diffusion direction.

7. The air diffuser according to claim 5, wherein the spaced plate is disposed so that air flowing in the diffusion flow passage impinges on the spaced plate at an acute angle.

8. The air diffuser according to claim 1, which includes a central plate, said outlet extending around a periphery of the central plate, with the diffusion flow passage extending conically outwardly from the central plate.

9. The air diffuser according to claim 5, wherein the spaced plate defines a central aperture that is generally aligned with a centre of the central plate, so that the spaced plate is generally in register with the outlet.
